(19) United States
(12) Patent Application Publication
(10) Pub. No.: US 2024/0424273 A1
(43) Pub. Date: Dec. 26, 2024

(54) Microneedle Mediated Intracochlear Delivery

(71) Applicant: The Trustees of Columbia University in the City of New York, New York, NY (US)

(72) Inventors: Anil K. Lalwani, New York, NY (US); Jeffrey W. Kysar, New York, NY (US); Aykut Aksit, New York, NY (US)

(21) Appl. No.: 18/826,257

(22) Filed: Sep. 6, 2024

Related U.S. Application Data
(63) Continuation of application No. PCT/US2023/063853, filed on Mar. 7, 2023.
(60) Provisional application No. 63/317,175, filed on Mar. 7, 2022.
(60) Provisional application No. 63/336,153, filed on Apr. 28, 2022.

(51) Int. Cl. A61M 37/00

(52) U.S. Cl. CPC A61M 37/0015; A61M 2037/0023; A61M 2037/0061; A61M 2210/0662

(57) ABSTRACT

A method for injecting a therapeutic into the inner ear of a subject comprising providing a microneedle having a sharpened distal tip and a shaft with a maximum outer diameter of about 75 microns to about 150 microns, the microneedle defining an inner lumen with a diameter about 15 microns to about 50 microns; the inner lumen having a curvature near the needle tip such that the lumen opened at the side of the needle proximal the needle tip; the microneedle mounted on a blunt metallic syringe needle; the blunt metallic syringe needle attached to a syringe secured to a micropump; advancing the microneedle into the middle ear space and the perforating the round window membrane (RWM) with the needle tip; extending the needle such that the inner lumen is disposed in the inner ear space; injecting a volume of therapeutic into the inner ear space; and retracting of the microneedle from the RWM.

### CROSS-REFERENCE TO RELATED APPLICATIONS

This application is a Continuation of International Application PCT/US2023/063853, “Microneedle Mediated Intracochlear Delivery” (filed Mar. 7, 2023); which claims priority to U.S. Provisional Application 63/317,175, “Microneedle Mediated Intracochlear Delivery” (filed Mar. 7, 2022) and to U.S. Provisional Application 63/336,153, “Microneedle Mediated Direct, Safe Intracochlear Injection of Artificial Perilymph and Fluorescent Dye” (filed Apr. 28, 2022). All foregoing applications are incorporated herein by reference in their entireties for any and all purposes.

### GOVERNMENT SUPPORT CLAUSE

This invention was made with government support under contract no. R01-DC014547, awarded by the National Institutes of Health. The government has certain rights in the invention.

### FIELD

The disclosed subject matter relates to a microneedle apparatus for perforation of anatomic tissues. More particularly, the subject matter relates to a microneedle mediated perforation of the round window membrane of a subject for injection of a therapeutic.

### BACKGROUND

As the only non-ossified portal into the cochlea, the RWM has been the target for many methods of minimally invasive intracochlear access. Drug-eluting cochlear implants containing dexamethasone or growth factors have been shown to be neuroprotective following implant insertion, and RWM implants such as the Hybrid Ear Cube can be used for treatment of inner ear disorders requiring long-term dosing of therapeutics. Though implants are effective and reliable for intracochlear access, they are often permanent and require significant lifestyle modification for long-term maintenance. Direct intracochlear injection through the RWM is a more transient means of drug administration to the cochlea, and studies have demonstrated greater concentrations compared to intratympanic injections. However, direct trauma to the RWM may produce hearing loss, and perilymphatic leakage through the resultant perforation is often significant. A methodology for minimally traumatic RWM perforation and intracochlear injection is thus highly warranted.

### SUMMARY

In one aspect, the disclosed subject matter provides a method for injecting a therapeutic into the inner ear of a subject including providing a microneedle having a sharpened distal tip and a shaft with a maximum outer diameter of about 75 microns to about 150 microns, the microneedle defining an inner lumen with a diameter about 15 microns to about 50 microns; the inner lumen having a curvature near the needle tip such that the lumen is opened at the side of the needle proximal the needle tip; the microneedle mounted on a blunt metallic syringe needle; the blunt metallic syringe needle attached to a syringe secured to a micropump; advancing the microneedle into the middle ear space and the perforating the round window membrane (RWM) with the needle tip; extending the needle such that the inner lumen is disposed in the inner ear space; injecting a volume of therapeutic into the inner ear space; and retracting of the microneedle from the RWM.

In some embodiments, the microneedle has a maximum outer diameter less than about 100 microns along a portion of the shaft. In some embodiments, the microneedle is fabricated from a biocompatible polymer, stainless steel, or titanium.

In some embodiments, the blunt metallic syringe needle is a 30 gauge syringe needle. In some embodiments, the inner lumen has a diameter of 35 μm or less.

In some embodiments, injecting a volume of therapeutic includes injecting 1.0 μL or less of therapeutic into the inner ear space. In some embodiments, injecting a volume of therapeutic comprises injecting 1.0 μL or less of therapeutic into the inner ear space with a rate of 1 μL/min or less.

In one aspect, the disclosed subject matter provides a method for injecting a therapeutic into the cochlea of a subject including providing a microneedle having a sharpened distal tip and a shaft with a maximum outer diameter of about 75 microns to about 150 microns, the microneedle defining an inner lumen with a diameter about 15 microns to about 50 microns; the inner lumen having a curvature near the needle tip such that the lumen is opened at the side of the needle proximal the needle tip; the microneedle mounted on a blunt metallic syringe needle; the blunt metallic syringe needle attached to a syringe secured to a micropump; advancing the microneedle into the middle ear space and the perforating the round window membrane (RWM) with the needle tip; extending the needle through the RWM such that the inner lumen is disposed in the inner ear; injecting volume of therapeutic into the cochlea to distribute the therapeutic into the basal, middle and apical turns of the cochlea; and retracting of the microneedle from the RWM.

In some embodiments, the microneedle has a maximum outer diameter less than about 100 microns along a portion of the shaft. In some embodiments, the microneedle is fabricated from a biocompatible polymer, stainless steel, or titanium.

In some embodiments, the blunt metallic syringe needle is a 30 gauge syringe needle. In some embodiments, the inner lumen has a diameter of 35 μm or less.

In some embodiments, injecting a volume of therapeutic includes injecting 1.0 μL or less of therapeutic into the inner ear space. In some embodiments, injecting a volume of therapeutic comprises injecting 1.0 μL or less of therapeutic into the inner ear space with a rate of 1 μL/min or less.

In another aspect, a system for injecting a therapeutic into the inner ear of a subject is provided including a microneedle having a sharpened distal tip and a shaft with a maximum outer diameter of about 75 microns to about 150 microns, the microneedle defining an inner lumen with a diameter about 15 microns to about 50 microns; the inner lumen having a curvature near the needle tip such that the lumen opened at the side of the needle proximal the needle tip; the microneedle mounted on a blunt metallic syringe needle; the blunt metallic syringe needle attached to a syringe secured to a micropump configured to inject 1.0 μL or less of therapeutic into the inner ear space with a rate of 1 μL/min or less.

In some embodiments, the microneedle has a maximum outer diameter less than about 100 microns along a portion of the shaft. In some embodiments, the microneedle is fabricated from a biocompatible polymer, stainless steel, or titanium.

In some embodiments, the blunt metallic syringe needle is a 30 gauge syringe needle. In some embodiments, the inner lumen has a diameter of 35 μm or less.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a scanning electron microscope image of a single-lumen hollow microneedle. A 100-μm scale bar is displayed.

FIG. 2A is a confocal image of RWMs 48 hours after microneedle-mediated injection of 1.0 μL of artificial perilymph.

FIG. 2B is a confocal image of RWMs 48 hours after microneedle-mediated injection of 2.5 μL of artificial perilymph.

FIG. 2C is a confocal image of RWMs 48 hours after microneedle-mediated injection of 5.0 μL of artificial perilymph.

FIG. 3A illustrates mean CAP thresholds immediately preceding microneedle-mediated injection of artificial perilymph (blue line) and 48 hours following injection (red line), for 1.0 μL injections.

FIG. 3B illustrates mean CAP thresholds immediately preceding microneedle-mediated injection of artificial perilymph (blue line) and 48 hours following injection (red line), for 2.5 μL injections.

FIG. 3C illustrates mean CAP thresholds immediately preceding microneedle-mediated injection of artificial perilymph (blue line) and 48 hours following injection (red line), for 5.0 μL injections.

FIG. 4A illustrates mean DPOAEs immediately preceding microneedle-mediated injection of artificial perilymph (blue line), 1 hour following injection (red line), and 48 hours following injection (green line), for 1.0 μL injections.

FIG. 4B illustrates mean DPOAEs immediately preceding microneedle-mediated injection of artificial perilymph (blue line), 1 hour following injection (red line), and 48 hours following injection (green line), for 2.5 μL injections.

FIG. 4C illustrates mean DPOAEs immediately preceding microneedle-mediated injection of artificial perilymph (blue line), 1 hour following injection (red line), and 48 hours following injection (green line), for 5.0 μL injections.

FIG. 5A illustrates mean CAP thresholds immediately preceding microneedle-mediated injection of 1.0 μL of 70 μM FM 1-43 FX (blue line) and 48 hours following injection (red line)

FIG. 5B illustrates mean DPOAEs immediately preceding microneedle-mediated injection of 1.0 μL of 70 μM FM 1-43 FX (blue line), 1 hour following injection (red line), and 48 hours following injection (green line). Shaded areas represent 95% confidence intervals. All DPOAEs remain above the noise level; significant differences are noted for several frequencies in the low-middle range.

FIG. 6A is a confocal image of outer hair cells from the basal turns of cochleae injected with 1.0 μL of 70 μM FM 1-43 FX, 10× magnification.

FIG. 6B is a confocal image of outer hair cells from the middle turns of cochleae injected with 1.0 μL of 70 μM FM 1-43 FX, 10× magnification.

FIG. 6C is a confocal image of outer hair cells from the apical turns of cochleae injected with 1.0 μL of 70 μM FM 1-43 FX, 10× magnification.

FIG. 6D is a confocal image of outer hair cells from the basal turn of a contralateral cochlea, 10× magnification.

FIG. 6E illustrates average relative fluorescence intensities from cochleae injected with 1.0 μL of 70 μM FM 1-43 FX, divided into basal, middle, and apical turns.

FIG. 6F illustrates P-values from repeated measures ANOVA tests and paired t-tests comparing relative fluorescence intensity of the basal, middle, and apical turns

### DETAILED DESCRIPTION OF EXEMPLARY EMBODIMENTS

It is to be understood that both the foregoing general description and the following detailed description are exemplary and explanatory only, and are not restrictive of the invention, as claimed. In this description, the use of the singular includes the plural, the word “a” or “an” means “at least one,” and the use of “or” means “and/or,” unless specifically stated otherwise. Furthermore, the use of the term “including,” as well as other forms, such as “includes” and “included” is not limiting. Also, terms such as “element” or “component” encompass both elements or components comprising one unit and elements or components that comprise more than one unit unless specifically stated otherwise.

Use of the term “about,” when used with a numerical value, is intended to include +/−10%. For example, if a dimension is identified as about 200, this would include 180 to 220 (plus or minus 10%).

The terms “patient,” “individual,” and “subject” are used interchangeably herein, and refer to a mammalian subject to be treated, with human patients being preferred. In some cases, the methods of the invention find use in experimental animals, in veterinary application, and in the development of animal models for disease.

In an exemplary embodiment, of the disclosed subject matter, a system for injecting a therapeutic into the inner ear of a subject is provided including a microneedle having a sharpened distal tip and a shaft with a maximum outer diameter of about 75 microns to about 150 microns, the microneedle defining an inner lumen with a diameter about 15 microns to about 50 microns; the inner lumen having a curvature near the needle tip such that the lumen opened at the side of the needle proximal the needle tip; the microneedle mounted on a blunt metallic syringe needle; the blunt metallic syringe needle attached to a syringe secured to a micropump configured to inject 1.0 μL or less of therapeutic into the inner ear space with a rate of 1 μL/min or less.

In an exemplary embodiment, a microneedle capable of reliably perforating the RWM with minimal effects on hearing and RWM anatomy is used herein. For example, two-photon polymerization lithography (2PP) was used to direct-write microneedles with varying length, sharpness, taper angle, diameter, and cross-sectional design. Microneedles manufactured using two-photon polymerization (2PP) lithography are fabricated with an acrylic-based resin, such as negative-tone resins, IP photoresins, e.g., IP-S Photoresist. In some embodiments, the microneedle is manufactured from ultra-high precision 3D molds made via 2PP lithography. Two-photon lithography can be used to manufacture molds for making thermoplastic microneedles and needle arrays for drug delivery and fluid sampling across the anatomic membranes the ear, eye and the CNS such as the RWM. Since the precision of this manufacturing process is very high, very smooth ultra-sharp needles. It is understood that an array of multiple microneedles, such as two microneedle, three microneedles, or more microneedles can be used in the disclosure described herein, can be made that are specifically engineered to reduce insertion force, minimizing the damage to the tissue in question and any surrounding tissue. In some embodiments, biocompatible polymers, stainless steel, or titanium can be used to manufacture the microneedle. The microneedle 100 can be a hollow microneedle mounted on an enlarged base portion 102, a having a substantially cylindrical shaft 104, a substantially conical tapered portion 106 and a sharpened tip portion 108. The microneedle can be provided with one or more interior lumens 11, each of the lumens can be used for injection/introduction or aspiration of fluid. In some embodiments, the lumens are in in fluid communication with an interior portion of the tubing connected to a syringe pump.

In an exemplary embodiment, the shaft 106 has a diameter of 100 μm. In some embodiments, the shaft has a diameter in the range of 50 μm to 200 μm. Exemplary diameter dimensions of the shaft 106 of microneedle 104 include 50 μm, 60 μm, 75 μm, 90 μm, 100 μm, 110 μm, 125 μm, 1400 μm, 150 μm, 175 μm 200 μm and any dimensions inclusive. In an exemplary embodiment, the needle length is 475 μm. In some embodiments, the needle length is 250 μm, 275 μm, 300 μm, 350 μm, 400 μm, 450 μm, 475 μm, 500 μm, 550 μm, 600 μm, 650 μm, 700 μm, 750 μm and any dimensions inclusive. In an exemplary embodiment, the diameter of the interior lumens 120a/102b is 30 μm. In some embodiments, diameter of the interior lumen 109 is 15 μm, 20 μm, 30 μm, 40 μm, 50 μm, 60 μm, 70 μm, 80 μm and any dimensions inclusive. In an exemplary embodiment, the sharpness of the needle is defined by a tip radius of 500 nm to 3 μm. In some embodiments, the tip radius is 500 nm, 600 nm, 700 nm, 800 nm, 900 nm, 1 μm, 2 μm, 3 μm and any dimensions inclusive Further details regarding the microneedle are disclosed in applications WO/2014/093875; U.S. Pat. No. 10,821,276; WO/2015/20092; U.S. Pat. No. 11,413,191; U.S. application Ser. No. 17/887,966; WO/2017/160948; US 2019/0200927; WO/2019/136133; US 2020/0345994; WO/2019/204760; US 2021/0045925; US 2022/0175413; WO/2021/050404; US 2022/0176096; WO/2020/214802; US 2022/0032023, PCT/US23/61767 and PCT/US23/61545 all of which are incorporated by reference in their entirety herein.

Puncture of the guinea pig RWM with 100-μm microneedles resulted in oval, lens-shaped perforations with a mean major axis of 95.9 μm and mean minor axis of 25.4 μm; perforations healed completely within 48-72 hours with no notable shifts in hearing thresholds. Importantly, incorporation of a 35-μm hollow lumen in the 100-μm microneedle allowed for aspiration of 1 μL of perilymph from the cochlea without changes in hearing; LC-MS proteomic characterization of the aspirate identified 620 guinea pig proteins, including the inner ear protein cochlin. In fact, proteomic characterization of microneedle-mediated aspirate was precise enough to identify changes in the proteome following corticosteroid administration, with the nerve growth factor VGF significantly upregulated after both intraperitoneal (IP) and intratympanic (IT) dexamethasone administration.

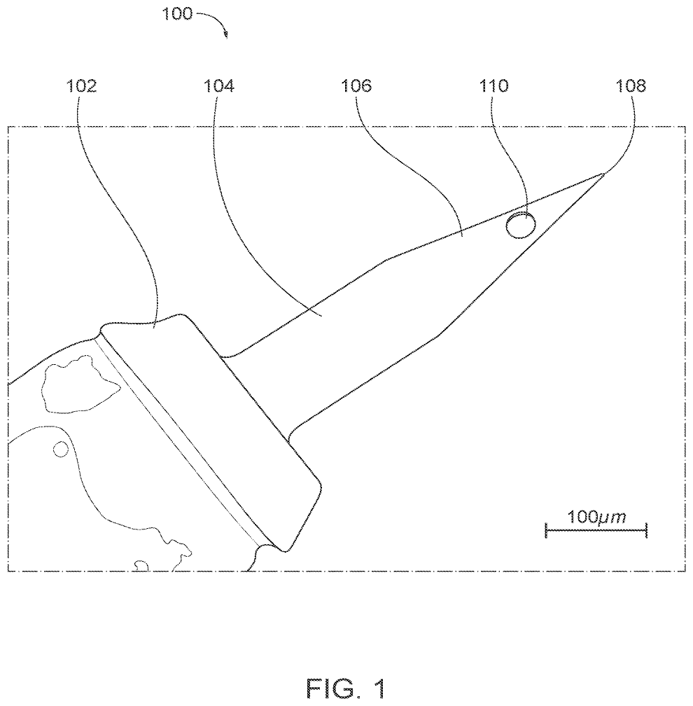

Microneedle design and synthesis for an exemplary embodiment is described herein. Microneedles were designed with SolidWorks software (Dassault Systems SolidWorks Corporation, Concord, NH) and stereolithography files were constructed using Describe software (Nanoscribe GmbH, Karlsruhe, Germany) at a slicing distance of 1 μm and laser intensity of 80%. Microneedles were then synthesized with the Photonic Professional GT 2PP system using photoresist IP-S (Nanoscribe GmbH). The outer diameter of the needle was set to 100 μm with inner diameter 35 μm. The inner lumen was placed centrally with curvature near the needle tip such that the lumen opened at the side of the needle tip (FIG. 1). Additionally, the outer diameter at the base of the needle was increased to 139 μm with inner diameter 95 μm so to decrease overall fluid resistance.

Direct intracochlear injection is described herein for an exemplary embodiment. A total of 20 Hartley guinea pigs weighing 200-300 g were relied upon for the trials; 15 were used for artificial perilymph injection and 5 were used for FM 1-43 FX injection. For artificial perilymph injections, animals were divided into 3 groups each receiving 1.0 μL, 2.5 μL, or 5.0 μL of artificial perilymph (each with n=5). All procedures were completed on the right ear. Guinea pigs were anesthetized with isoflurane gas (3.0% for induction, 1.0-3.0% for maintenance) and administered buprenorphine sustained release (0.1 mg/kg) and meloxicam (0.5 mg/kg) for analgesia. Lidocaine was also injected in the post-auricular area for local anesthesia. To reduce head movements from deep breaths, the head was fixed with a modular 3D-printed head holder consisting of two screws placed anterior to the external auditory meatus and posterior to the orbit17; lidocaine was injected at the screw fixation sites for local anesthesia.

A 1-cm post-auricular incision was made with a scalpel blade, and blunt dissection was used to expose the tympanic bulla and the facial nerve emerging from bone. A Stryker S2 πDrive drill (Stryker, Kalamazoo, MI) with a 1 mm drill tip was used to open the bulla, and fine forceps were used to remove bone and form a 2-3 mm opening into the middle ear space.

A hollow microneedle, mounted on a 2-inch, 30-gauge, blunt, small hub removable needle (Hamilton Company, Reno, NV), was attached to a 10 μL Gastight Hamilton syringe (Model 1701 RN, Hamilton Company, Reno, NV); the syringe in turn was secured to a UMP3 UltraMicroPump (World Precise Instruments, Sarasota, FL), which was fixed to a micromanipulator. Using the micromanipulator, the hollow microneedle was advanced into the middle ear space and the RWM was perforated. Downward RWM displacement with initial microneedle contact was noted, followed by upward deflection of the RWM upon microneedle entry into the inner ear space. For animals receiving artificial perilymph (NaCl 120 mM, KCl 3.5 mM, CaCl2 1.5 mM, glucose 5.5 mM, HEPES 20 mM, NaOH to pH=7.5), 1.0 μL, 2.5 μL, or 5.0 μL of artificial perilymph was injected into the inner ear space with a rate of 1 μL/min. For animals receiving FM 1-43 FX (FM 1-43 FX, fixable analog of FM 1-43 membrane stain, Thermo Fischer Scientific), 1.0 μL of 70 μM FM 1-43 FX in artificial perilymph was injected into the cochlea at a rate of 1 μL/min. Upon retraction of the microneedle from the RWM, the lens-shaped perforation on the RWM was visualized as confirmation.

Following perforation, simple interrupted sutures were used to close the postauricular incision, and guinea pigs were returned to the animal facility for recovery. Guinea pigs were euthanized with phenytoin/pentobarbital overdose 48 hours after RWM perforation. For animals receiving artificial perilymph injection, cochleae were harvested and stained with 1 mM rhodamine B, a fluorescent stain for elastic tissue, for 5 minutes and rinsed with PBS. For animals receiving FM 1-43 FX, cochleae were harvested, fixed for 24 hours in 4% formalin injected into the cochlea through the RWM, washed with PBS, and decalcified for 7-10 days in 0.15 M EDTA. Decalcified cochleae were subsequently dissected into three pieces representing the basal, middle, and apical turns and mounted on glass slides using VECTASHIELD antifade mounting medium. One contralateral cochlea was also harvested and treated in the same fashion.

Audiometric tests were performed. Compound action potential (CAP) and distortion product otoacoustic emissions (DPOAE) were used to evaluate hearing as previously described. CAP was measured immediately prior to perforation, while DPOAE was measured prior to perforation and 1 hour following perforation. Both CAP and DPOAE were measured 48 hours following injection. All hearing tests were completed under anesthesia.

CAP measurements summate individual action potentials at the cochlear base following sound stimulation, thereby measuring the activity of the cochlear nerve. Tone pips were played to the right ear and a silver electrode was used to measure electrical activity at the cochlear base, with a reference electrode placed in nearby fascia and a ground electrode inserted in the skin by the scapular ridge. CAP responses were measured for a total of 18 frequencies ranging from 0.5 kHz to 40 kHz. Tone intensities were increased at 5 dB SPL increments, and the lowest amplitude to produce a discernable response curve was identified as the hearing threshold. Experimenters were blinded to all previous threshold measurements to reduce bias. Two-tailed paired t-tests with a significance level of 0.05 were used to evaluate for significant changes.

DPOAE measurements are responses produced by cochlear outer hair cells upon stimulation by two pure tone frequencies played simultaneously. Thus, DPOAE measurements assess the health of outer hair cells as a proxy for potential hearing loss. An ear tube containing a speaker and a low-noise Sokolich ultrasonic probe microphone was placed in the external auditory canal, and simultaneous sound stimuli were played at 70 and 80 dB SPL. Stimuli had a fixed frequency ratio of f2/f1=1.2 and ranged from 1 kHz to 32 kHz at 1 KHz increments. Distortion products were detected by the microphone, and DPOAE measurements with 2f1−f2=3 dB above the noise floor level were identified as positive responses. Repeated measures ANOVA tests, followed by pairwise two-tailed paired t-tests with a significance level of 0.05, were used to evaluate for significant changes.

Confocal imaging and quantification: Confocal microscopy was used to assess RWM healing following artificial perilymph injection and hair cell distribution following FM 1-43 FX injection. A Nikon A1R laser-scanning confocal microscopy (Nikon Instruments, Melville, NY) was used to acquire z-stack images through RWMs and basal, middle, and apical turns of the cochlea. For RWMs, images were obtained using a Nikon 10× Plan Apo VC (0.45 NA) objective with 1.0 optical zoom, with pixel size 1.2×1.2 μm and 5 μm step size. Samples were scanned with a 561 nm laser line with a pixel dwell time of 1.1 μs, and emitted light at 570-620 nm was allowed into the detector. Maximum intensity projection images were generated from z-stacks using NIS-Elements (Nikon) to visualize RWMs and examine for residual scarring or inflammation. For cochlear turns, images were obtained using the same objective with pixel size 1.2×1.2 μm and 1 μm step size. Samples were scanned with a 478 nm laser line with a pixel dwell time of 1.1 μs, and emitted light at 570-620 nm was allowed into the detector. Maximum intensity projection images were generated from z-stacks using NIS-Elements (Nikon), and fluorescence intensity of outer hair cell rows was quantified using Fiji (ImageJ, National Institutes of Health, Bethesda, MD). Specifically, outer hair cell layers were traced over the entirety of the imaged sample, and the mean grey value, or pixel brightness, for the trace was measured. Measured fluorescence intensity values for each cochlea were normalized based on the intensity value obtained at the basal turn, with normalized intensity=measured intensity/measured intensity at basal turn. Repeated measures ANOVA tests, followed by pairwise two-tailed paired t-tests with a significance level of 0.05 were used to evaluate for significant changes. Inner hair cell fluorescence intensity was not quantified due to interference from surrounding structures.

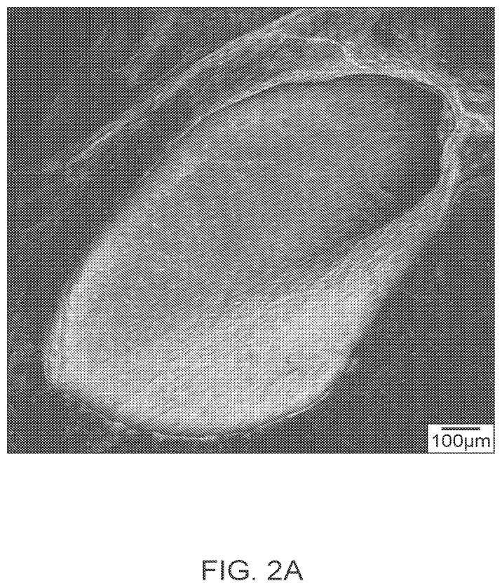

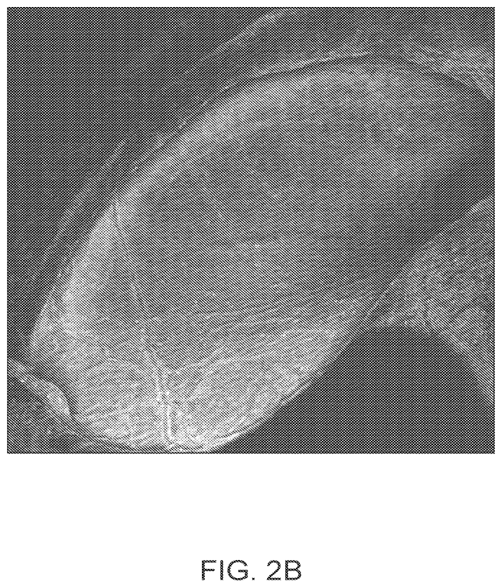

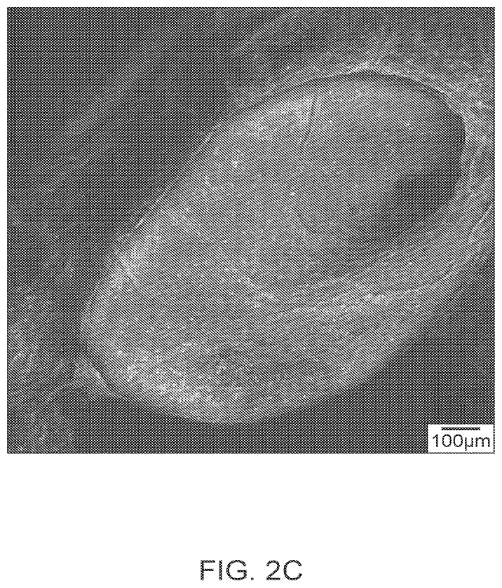

Results of the above-described methods of microneedle-mediated intracochlear injection of artificial perilymph are noted herein. CAP and DPOAE data were obtained for all 15 animals undergoing microneedle-mediated injection of artificial perilymph. Perforations were well-visualized under light microscopy for all 15 animals immediately following microneedle-mediated injection; self-limiting perilymph efflux from the perforation site was also noted for all 15 animals. Confocal imaging of RWMs harvested 48 hours following injection revealed full reconstitution with no residual perforation, inflammation, or scarring. Examples of healed RWMs are displayed in FIGS. 2A, 2B, and 2C, for 1.0 μL, 2.5 μL, and 5.0 μL injections, respectively.

FIGS. 2A-C are confocal images of RWMs 48 hours after microneedle-mediated injection of 1.0 μL (FIG. 2A), 2.5 μL (FIG. 2B), and 5.0 μL (FIG. 2C) of artificial perilymph at 10× magnification. A 100-μm scale bar is displayed, corresponding to the diameter of the microneedle. No residual perforation, inflammation, or scarring is noted in any of the images.

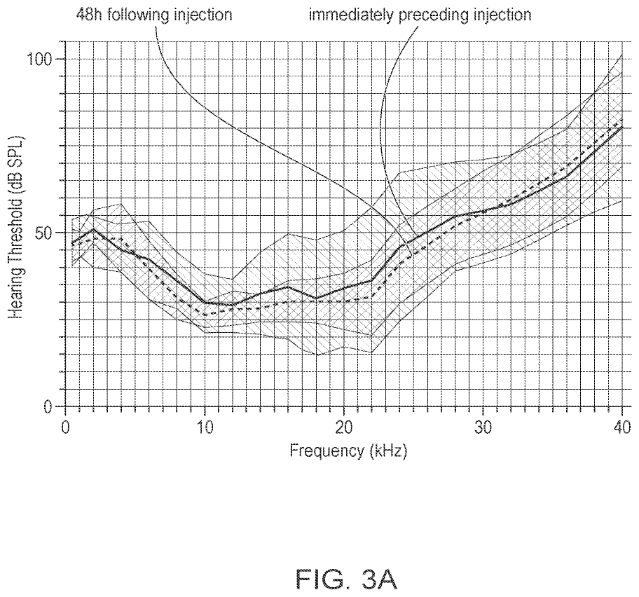

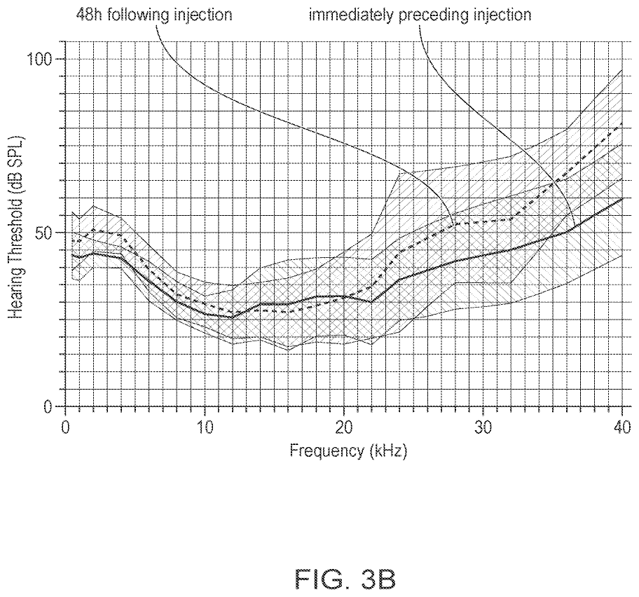

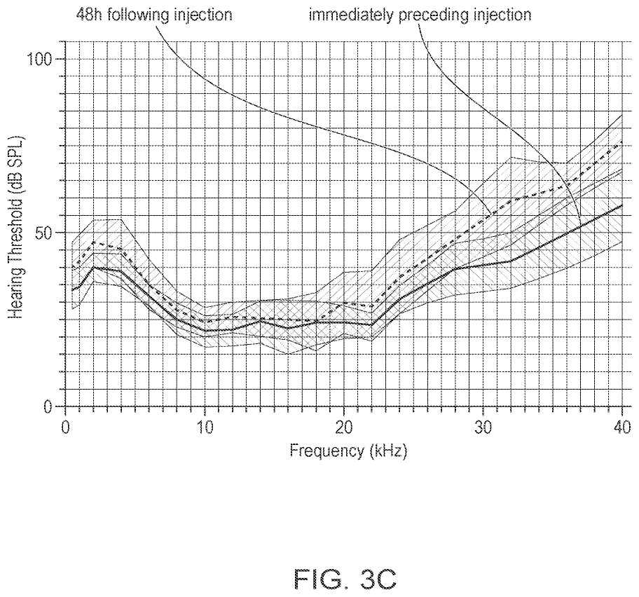

FIGS. 3A-C illustrate, ean CAP thresholds immediately preceding microneedle-mediated injection of artificial perilymph (blue line) and 48 hours following injection (red line), for 1.0 μL (FIG. 3A), 2.5 μL (FIG. 3B), and 5.0 μL (FIG. 3C) injections. Shaded areas represent 95% confidence intervals. No threshold shifts are observed for 1.0 μL injection; predominantly high-frequency threshold shifts are observed for 2.5 μL and 5.0 μL injections.

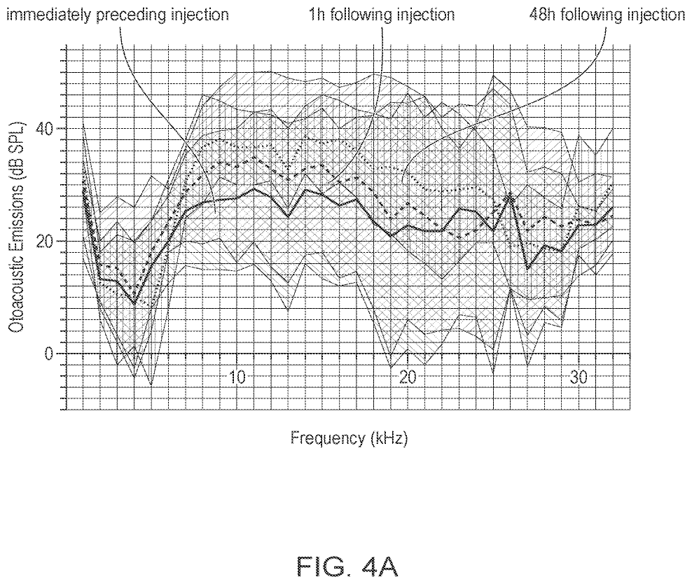

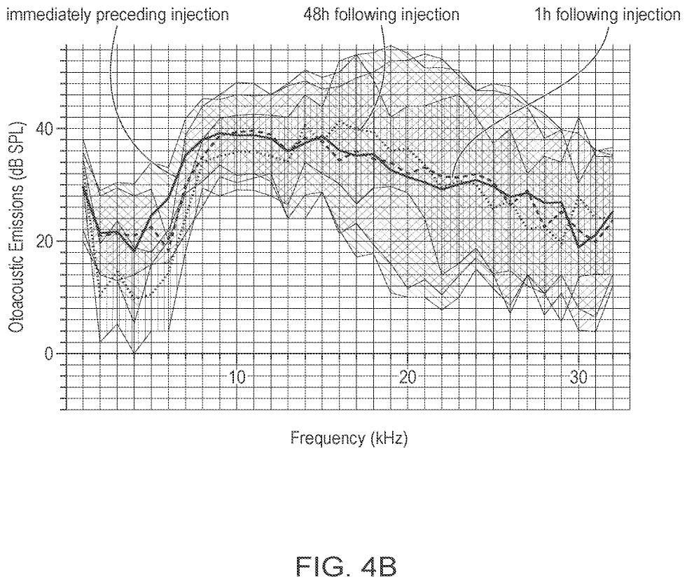

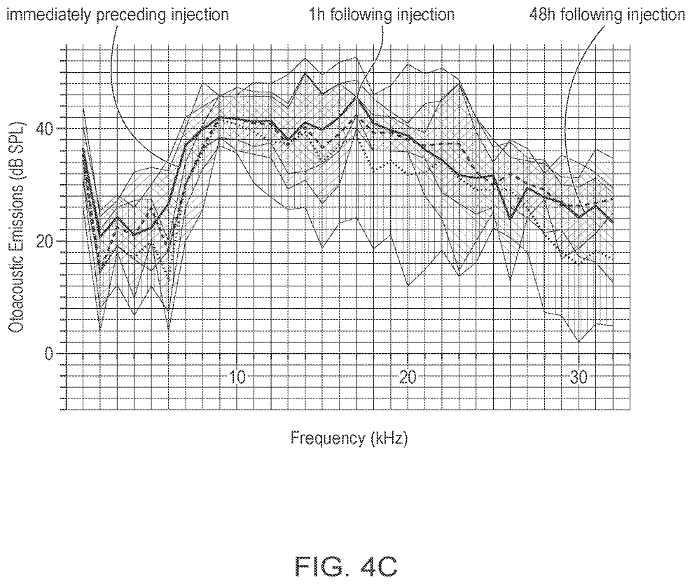

FIGS. 4A-C illustrate mean DPOAEs immediately preceding microneedle-mediated injection of artificial perilymph (blue line), 1 hour following injection (red line), and 48 hours following injection (green line), for 1.0 μL (FIG. 4A), 2.5 μL (FIG. 4B), and 5.0 μL (FIG. 4C) injections. Shaded areas represent 95% confidence intervals. All DPOAEs remain above the noise level and no significant differences are noted for any of the presented frequencies.

For animals receiving 1.0 μL of artificial perilymph, there were no significant CAP threshold shifts at any tested frequencies 48 hours following perforation (FIG. 3A), with p>0.05 at all tested frequencies (Table 1). Table 1 illustrates P-values from paired t-tests comparing pre-perforation CAP and 48-hour post-perforation CAP for microneedle-mediated injection of 1.0 μL, 2.5 μL, and 5.0 μL of artificial perilymph. Significant values are bolded

TABLE 1

| Frequency (KHz) | 1.0 μL (t-test) | 2.5 μL (t-test) | 5.0 μL (t-test) |
|---|---|---|---|
| 0.5 | 0.88 | 0.17 | 0.025 |
| 1 | 0.58 | 0.069 | 0.010 |
| 2 | 0.52 | 0.031 | 0.015 |
| 4 | 0.31 | 0.058 | 0.078 |
| 6 | 0.34 | 0.35 | 0.22 |
| 8 | 0.15 | 0.58 | 0.15 |
| 10 | 0.36 | 0.50 | 0.38 |
| 12 | 0.72 | 0.77 | 0.0061 |
| 14 | 0.20 | 0.77 | 0.68 |
| 16 | 0.38 | 0.74 | 0.52 |
| 18 | 0.87 | 0.60 | 0.96 |
| 20 | 0.56 | 0.91 | 0.16 |
| 22 | 0.53 | 0.50 | 0.23 |
| 24 | 0.56 | 0.33 | 0.20 |
| 28 | 0.73 | 0.09 | 0.14 |
| 32 | 0.79 | 0.23 | 0.0039 |
| 36 | 0.55 | 0.025 | 0.0054 |
| 40 | 0.74 | 0.033 | 0.00036 |

Additionally, DPOAEs remained above the noise floor for all tested frequencies (FIG. 4A). There was one significant change in DPOAE at 28 kHz (p=0.012), with pairwise t-testing revealing higher DPOAE 1 hour post-perforation compared to pre-perforation (p=0.0096); all other tested frequencies did not reveal significant differences in DPOAEs obtained pre-perforation, 1 hour post-perforation, and 48 hours post-perforation (See Table 2A). Tables 2A-C show P-values from repeated measures ANOVA tests and paired t-tests comparing pre-perforation DPOAE, 1-hour post-perforation DPOAE, and 48-hour post-perforation DPOAE for microneedle-mediated injection of 1.0 μL (Table 2A), 2.5 μL (Table 2B) and 5.0 μL (Table 2C), of artificial perilymph. Significant ANOVA values, with their corresponding t-tests, are bolded.

TABLE 2A

| Frequency | | t-test | t-test | t-test |
|---|---|---|---|---|
| (kHz) | ANOVA | (pre vs. 1 h) | (pre vs. 48 h) | (1 h vs. 48 h) |
| 1 | 0.60 | 0.33 | 0.27 | 0.27 |
| 2 | 0.88 | 0.43 | 0.84 | 0.48 |
| 3 | 0.57 | 0.65 | 0.77 | 0.62 |
| 4 | 0.62 | 0.63 | 0.82 | 0.95 |
| 5 | 0.71 | 0.41 | 0.23 | 0.23 |
| 6 | 0.92 | 0.25 | 0.80 | 0.38 |
| 7 | 0.54 | 0.50 | 0.38 | 0.67 |
| 8 | 0.59 | 0.27 | 0.19 | 0.43 |
| 9 | 0.10 | 0.24 | 0.18 | 0.52 |
| 10 | 0.15 | 0.34 | 0.26 | 0.65 |
| 11 | 0.078 | 0.34 | 0.34 | 0.78 |
| 12 | 0.091 | 0.32 | 0.23 | 0.55 |
| 13 | 0.074 | 0.19 | 0.29 | 0.79 |
| 14 | 0.20 | 0.33 | 0.16 | 0.38 |
| 15 | 0.21 | 0.21 | 0.30 | 0.59 |
| 16 | 0.31 | 0.24 | 0.12 | 0.28 |
| 17 | 0.20 | 0.25 | 0.11 | 0.39 |
| 18 | 0.29 | 0.11 | 0.16 | 0.45 |
| 19 | 0.19 | 0.45 | 0.18 | 0.26 |
| 20 | 0.29 | 0.25 | 0.41 | 0.56 |
| 21 | 0.26 | 0.25 | 0.52 | 0.64 |
| 22 | 0.16 | 0.74 | 0.56 | 0.54 |
| 23 | 0.14 | 0.078 | 0.71 | 0.36 |
| 24 | 0.15 | 0.31 | 0.66 | 0.45 |
| 25 | 0.20 | 0.46 | 0.64 | 0.83 |
| 26 | 0.16 | 0.57 | 0.12 | 0.15 |
| 27 | 0.11 | 0.15 | 0.63 | 0.84 |
| 28 | 0.012 | 0.0096 | 0.93 | 0.41 |
| 29 | 0.20 | 0.20 | 0.99 | 0.44 |
| 30 | 0.44 | 0.53 | 0.49 | 0.63 |
| 31 | 0.37 | 0.99 | 0.49 | 0.27 |
| 32 | 0.71 | 0.46 | 0.22 | 0.045 |

For animals receiving 2.5 μL of artificial perilymph, there were significant CAP threshold shifts at 2 kHz (7 dB, p=0.031), 36 kHz (16.8 dB, p=0.025), and 40 kHz (21.8 dB, p=0.033) 48 hours following perforation (FIG. 3B) (Table 1). DPOAEs remained above the noise floor for all tested frequencies (FIG. 4B), and there were no significant differences in DPOAEs obtained pre-perforation, 1 hour post-perforation, and 48 hours post-perforation, for all tested frequencies (See Table 2B).

TABLE 2B

| Frequency | | t-test | t-test | t-test |
|---|---|---|---|---|
| (kHz) | ANOVA | (pre vs. 1 h) | (pre vs. 48 h) | (1 h vs. 48 h) |
| 1 | 0.20 | 0.81 | 0.15 | 0.25 |
| 2 | 0.13 | 0.73 | 0.0038 | 0.0049 |
| 3 | 0.99 | 0.75 | 0.18 | 0.18 |
| 4 | 0.35 | 0.29 | 0.16 | 0.028 |
| 5 | 0.40 | 0.54 | 0.054 | 0.017 |
| 6 | 0.99 | 0.0093 | 0.047 | 0.36 |
| 7 | 0.12 | 0.019 | 0.035 | 0.23 |
| 8 | 0.37 | 0.036 | 0.32 | 0.83 |
| 9 | 0.88 | 0.63 | 0.40 | 0.49 |
| 10 | 0.94 | 0.66 | 0.49 | 0.50 |
| 11 | 0.91 | 0.41 | 0.53 | 0.48 |
| 12 | 0.67 | 0.30 | 0.56 | 0.44 |
| 13 | 0.87 | 0.98 | 0.76 | 0.73 |
| 14 | 0.56 | 0.60 | 0.60 | 0.63 |
| 15 | 0.76 | 0.34 | 0.79 | 0.95 |
| 16 | 0.53 | 0.18 | 0.45 | 0.34 |
| 17 | 0.47 | 0.66 | 0.44 | 0.50 |
| 18 | 0.29 | 0.66 | 0.56 | 0.37 |
| 19 | 0.31 | 0.70 | 0.70 | 0.75 |
| 20 | 0.43 | 0.84 | 0.48 | 0.47 |
| 21 | 0.53 | 0.38 | 0.54 | 0.92 |
| 22 | 0.71 | 0.39 | 0.94 | 0.72 |
| 23 | 0.82 | 0.58 | 0.92 | 0.92 |
| 24 | 0.88 | 0.45 | 0.85 | 0.61 |
| 25 | 0.89 | 0.62 | 0.21 | 0.12 |
| 26 | 0.94 | 0.33 | 0.94 | 0.89 |
| 27 | 0.95 | 0.83 | 0.12 | 0.13 |
| 28 | 0.47 | 0.27 | 0.20 | 0.96 |
| 29 | 0.85 | 0.69 | 0.071 | 0.24 |
| 30 | 0.32 | 0.37 | 0.014 | 0.26 |
| 31 | 0.43 | 0.47 | 0.64 | 0.54 |
| 32 | 0.47 | 0.17 | 0.89 | 0.92 |

For animals receiving 5.0 μL of artificial perilymph, there were significant CAP threshold shifts at 0.5 kHz (6.4 dB, p=0.025), 1 kHz (7.2 dB, p=0.010), 2 kHz (7.2 dB, p=0.015), 12 kHz (3.6 dB, p=0.0061), 32 kHz (17.2 dB, p=0.0039), 36 kHz (13.8 dB, p=0.0054), and 40 kHz (18.2 dB, p=0.00036) 48 hours following perforation (FIG. 3c) (Supplementary Table 1). DPOAEs remained above the noise floor for all tested frequencies (FIG. 4c). There was one significant change in DPOAE at 30 kHz (p=0.012), with pairwise t-testing revealing no significant differences; all other tested frequencies did not reveal significant differences in DPOAEs obtained pre-perforation, 1 hour post-perforation, and 48 hours post-perforation (See Table 2C).

TABLE 2C

| Frequency | | t-test | t-test | t-test |
|---|---|---|---|---|
| (kHz) | ANOVA | (pre vs. 1 h) | (pre vs. 48 h) | (1 h vs. 48 h) |
| 1 | 0.33 | 0.60 | 0.49 | 0.76 |
| 2 | 0.38 | 0.10 | 0.060 | 0.92 |
| 3 | 0.37 | 0.089 | 0.13 | 0.33 |
| 4 | 0.85 | 0.95 | 0.35 | 0.40 |
| 5 | 0.30 | 0.24 | 0.65 | 0.24 |
| 6 | 0.87 | 0.066 | 0.0047 | 0.41 |
| 7 | 0.61 | 0.084 | 0.0052 | 0.96 |
| 8 | 0.65 | 0.36 | 0.013 | 0.97 |
| 9 | 0.98 | 0.93 | 0.40 | 0.83 |
| 10 | 0.90 | 0.93 | 0.54 | 0.59 |
| 11 | 0.92 | 0.71 | 0.34 | 0.38 |
| 12 | 0.86 | 0.51 | 0.29 | 0.44 |
| 13 | 0.81 | 0.41 | 0.86 | 0.91 |
| 14 | 0.58 | 0.41 | 0.67 | 0.84 |
| 15 | 0.74 | 0.18 | 0.22 | 0.63 |
| 16 | 0.52 | 0.15 | 0.34 | 0.75 |
| 17 | 1 | 0.10 | 0.22 | 0.57 |
| 18 | 0.83 | 0.36 | 0.13 | 0.30 |
| 19 | 0.60 | 0.85 | 0.31 | 0.43 |
| 20 | 0.66 | 0.61 | 0.35 | 0.46 |
| 21 | 0.81 | 0.82 | 0.48 | 0.45 |
| 22 | 0.32 | 0.37 | 0.86 | 0.59 |
| 23 | 0.46 | 0.43 | 0.88 | 0.39 |
| 24 | 0.69 | 0.82 | 0.44 | 0.56 |
| 25 | 0.29 | 0.55 | 0.38 | 0.56 |
| 26 | 0.065 | 0.15 | 0.15 | 0.33 |
| 27 | 0.096 | 0.84 | 0.13 | 0.36 |
| 28 | 0.58 | 0.56 | 0.21 | 0.12 |
| 29 | 0.84 | 0.87 | 0.038 | 0.25 |
| 30 | 0.012 | 0.65 | 0.32 | 0.092 |
| 31 | 0.29 | 0.90 | 0.099 | 0.12 |
| 32 | 0.22 | 0.33 | 0.33 | 0.079 |

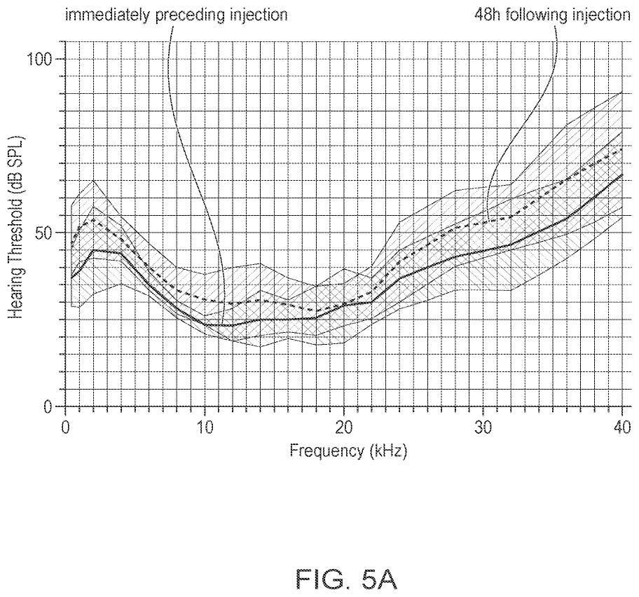

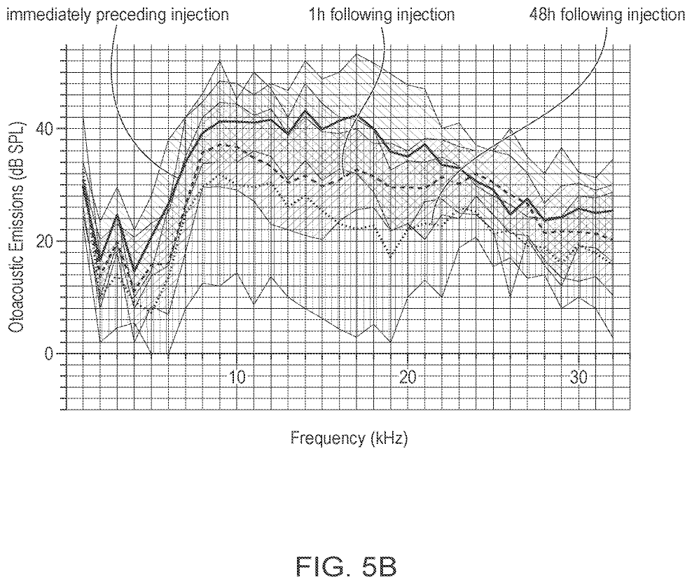

Microneedle-mediated intracochlear injection of FM 1-43 FX:CAP and DPOAE data were obtained for all five animals undergoing microneedle-mediated injection of FM 1-43 FX. A safe injection volume of 1.0 μL was chosen based on the artificial perilymph studies described above. Perforations were well-visualized under light microscopy for all five animals immediately following microneedle-mediated injection; self-limiting perilymph efflux from the perforation site was also noted for all five animals. There were no significant CAP threshold shifts at any tested frequencies 48 hours following FM 1-43 FX injection (FIG. 5A), with p>0.05 at all tested frequencies (Tables 3A-B). Additionally, DPOAEs remained above the noise floor for all tested frequencies (FIG. 5B).

FIG. 5A illustrates mean CAP thresholds immediately preceding microneedle-mediated injection of 1.0 μL of 70 μM FM 1-43 FX (blue line) and 48 hours following injection (red line). Shaded areas represent 95% confidence intervals. No threshold shifts are observed for any of the presented frequencies. FIG. 5B illustrates mean DPOAEs immediately preceding microneedle-mediated injection of 1.0 μL of 70 μM FM 1-43 FX (blue line), 1 hour following injection (red line), and 48 hours following injection (green line). Shaded areas represent 95% confidence intervals. All DPOAEs remain above the noise level; significant differences are noted for several frequencies in the low-middle range.

There were significant changes in DPOAE at 7 kHz (p=0.0014), 16 kHz (p=0.034), and 21 kHz (p=0.0038), with pairwise t-testing revealing lower DPOAE 1 hour post-perforation compared to pre-perforation for 7 kHz and 16 kHz (p=0.00081 and p=0.038, respectively), and lower DPOAE 48 hours post-perforation compared to pre-perforation for 21 kHz (p=0.037) (See Tables 3A-B). Table 3A shows P-values from paired t-tests comparing pre-perforation CAP and 48-hour post-perforation CAP for microneedle-mediated injection of 1.0 μL of 70 μM FM 1-43 FX. Table 3B shows P-values from repeated measures ANOVA tests and paired t-tests comparing pre-perforation DPOAE, 1-hour post-perforation DPOAE, and 48-hour post-perforation DPOAE for microneedle-mediated injection of 1.0 μL of 70 μM FM 1-43 FX. Significant ANOVA values, with their corresponding t-tests, are bolded.

TABLE 3A

| Frequency (KHz) | p-value |
|---|---|
| 0.5 | 0.18 |
| 1 | 0.13 |
| 2 | 0.31 |
| 4 | 0.40 |
| 6 | 0.15 |
| 8 | 0.14 |
| 10 | 0.072 |
| 12 | 0.28 |
| 14 | 0.40 |
| 16 | 0.36 |
| 18 | 0.75 |
| 20 | 0.94 |
| 22 | 0.53 |
| 24 | 0.54 |
| 28 | 0.29 |
| 32 | 0.35 |
| 36 | 0.29 |
| 40 | 0.49 |

TABLE 3B

| Frequency | | t-test | t-test | t-test |
|---|---|---|---|---|
| (kHz) | ANOVA | (pre vs. 1 h) | (pre vs. 48 h) | (1 h vs. 48 h) |
| 1 | 0.80 | 0.076 | 0.42 | 0.62 |
| 2 | 0.40 | 0.077 | 0.032 | 0.13 |
| 3 | 0.37 | 0.070 | 0.092 | 0.25 |
| 4 | 1 | 0.40 | 0.23 | 0.69 |
| 5 | 0.82 | 0.022 | 0.036 | 0.16 |
| 6 | 0.16 | 0.0076 | 0.18 | 0.73 |
| 7 | 0.0014 | 0.00081 | 0.31 | 0.86 |
| 8 | 0.16 | 0.056 | 0.27 | 0.50 |
| 9 | 0.21 | 0.058 | 0.34 | 0.60 |
| 10 | 0.33 | 0.074 | 0.17 | 0.42 |
| 11 | 0.30 | 0.090 | 0.23 | 0.62 |
| 12 | 0.61 | 0.11 | 0.20 | 0.78 |
| 13 | 0.21 | 0.12 | 0.15 | 0.64 |
| 14 | 0.095 | 0.043 | 0.16 | 0.76 |
| 15 | 0.24 | 0.044 | 0.18 | 0.69 |
| 16 | 0.034 | 0.038 | 0.089 | 0.45 |
| 17 | 0.097 | 0.087 | 0.086 | 0.30 |
| 18 | 0.071 | 0.061 | 0.099 | 0.31 |
| 19 | 0.26 | 0.30 | 0.089 | 0.16 |
| 20 | 0.18 | 0.36 | 0.11 | 0.18 |
| 21 | 0.0038 | 0.21 | 0.037 | 0.24 |
| 22 | 0.053 | 0.60 | 0.12 | 0.17 |
| 23 | 0.27 | 0.57 | 0.14 | 0.33 |
| 24 | 0.97 | 0.58 | 0.19 | 0.049 |
| 25 | 0.57 | 0.26 | 0.12 | 0.063 |
| 26 | 0.51 | 0.32 | 0.64 | 0.074 |
| 27 | 0.51 | 0.64 | 0.11 | 0.086 |
| 28 | 0.87 | 0.31 | 0.12 | 0.31 |
| 29 | 0.81 | 0.34 | 0.25 | 0.39 |
| 30 | 0.97 | 0.050 | 0.23 | 0.68 |
| 31 | 0.70 | 0.25 | 0.10 | 0.50 |
| 32 | 0.57 | 0.19 | 0.088 | 0.48 |

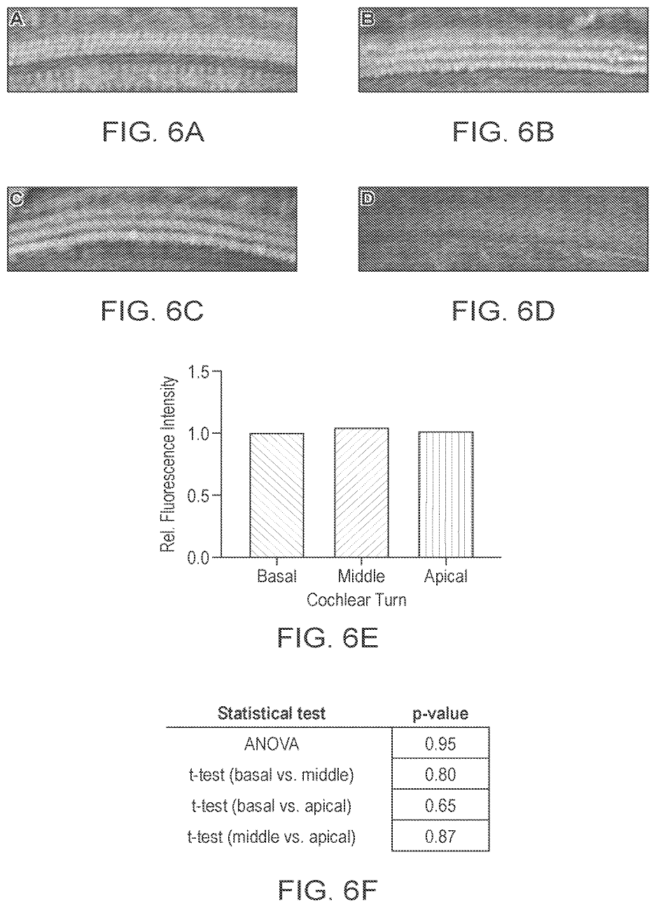

Confocal imaging of dissected cochleae fixed 48 hours following FM 1-43 FX injection revealed fluorescence throughout the basal, middle, and apical turns (FIGS. 6A-C) with definition of outer hair cells. In contrast, little fluorescence was noted in the basal turn of a contralateral cochlea (FIG. 6D). Inner hair cells were difficult to visualize due to overlapping fluorescence from surrounding structures. There was no significant difference in fluorescence intensity between the basal, middle, and apical turns (FIG. 6E-F).

FIGS. 6.A-C are confocal images of outer hair cells from the basal (FIG. 6A), middle (FIG. 6B), and apical (FIG. 6C) turns of cochleae injected with 1.0 μL of 70 μM FM 1-43 FX, 10× magnification. FIG. 6D illustrates outer hair cells from the basal turn of a contralateral cochlea, 10× magnification. FIG. 6E illustrates average relative fluorescence intensities from cochleae injected with 1.0 μL of 70 μM FM 1-43 FX, divided into basal, middle, and apical turns. FIG. 6F illustrates P-values from repeated measures ANOVA tests and paired t-tests comparing relative fluorescence intensity of the basal, middle, and apical turns. There is no significant difference in intensity between the imaged turns.

Here, we demonstrate that microneedle-mediated injection of up to 1.0 μL of injectate is safe and effective for intracochlear delivery of agents. Injection of 1.0 μL of artificial perilymph does not impair hearing and allows for full reconstitution of the RWM; additionally, injection of 1.0 μL of FM 1-43 FX produces fluorescence throughout the basal, middle, and apical turns of the cochlea within 48 hours of injection. These results support the use of microneedles for intracochlear drug delivery, most notably for precision therapeutics like gene therapy. A reliable and innocuous means of accessing the cochlea will thus promote the advancement of new therapies for hearing disorders, many of which are nearly impossible to treat due to the inaccessibility of the inner ear.

Although 1.0 μL of artificial perilymph may be injected without anatomic or physiologic consequences on the inner ear, injection of 2.5 μL or 5.0 μL results in moderate hearing loss in the high frequencies (32-40 KHz range) and mild hearing loss in the low frequencies (0.5-2 kHz range) based on CAP. For all injections, confocal imaging of the RWM revealed no residual perforation, scarring, or inflammation, indicating that loss of RWM integrity was not the cause of hearing loss for higher volume injections. Given that the total volume of the guinea pig cochlea is close to 10 μL, we suggest that injection of 2.5-5 μL, or 25-50% of the cochlear volume, increases pressure in the inner ear to an intolerable level, leading to damage of critical structures like the basilar or tectorial membranes. Interestingly, DPOAEs for all three injection volumes were relatively unremarkable, suggesting that hair cell health was preserved despite increases in pressure. In guinea pigs, the cochlear aqueduct is typically patent and plays some role in transmitting excessive pressure buildup to the cerebrospinal fluid (CSF) space; however, at volumes exceeding 25% of the total cochlear volume, this pressure relief is likely insufficient to prevent damage to hearing structures. In humans, the cochlear aqueduct is typically closed, which implies that the human inner ear is likely more sensitive to pressure changes than the guinea pig inner ear. As a result, intracochlear injection of 1.0 μL in humans may not be tolerable, but future studies on human cochleae are required to confirm this hypothesis. A cochleostomy in the cochlear apex may be introduced to relieve the buildup of pressure following microneedle-mediated injection, but the authors do not believe that this technique is safe for translation into human subjects.

An injection volume of 1.0 μL was found to be sufficient to produce significant distribution of FM 1-43 FX throughout the cochlea within 48 hours of injection. Notably, we find no significant difference in distribution between the basal, middle, and apical turns of the cochlea, in direct contrast to previous studies which report a strong basal-apical gradient persisting 72 hours after injection. Without being held to specific theory, it is possible that microneedle-mediated injection through the RWM produces a systemic uptake of FM 1-43 FX, which would nullify the basal-apical gradient as FM 1-43 FX diffuses back into the cochlea in an apical-basal fashion. An additional explanation for the lack of a basal-apical gradient is the reduced amount of fixative reaching the apical turn of the cochlea, leading to greater cell death in the apex compared to the base; previously literature has reported increased autofluorescence during the process of cell death.

CAP thresholds did not significantly change 48 hours after FM 1-43 FX injection; however, DPOAEs at a number of frequencies were significantly decreased following injection. The fact that CAP thresholds did not significantly increase may demonstrate that hair cell toxicity from FM 1-43 FX is relatively mild; hair cell loss seen in other studies may have been a product of the technique used to deliver fluorescent agent to the cochlea (i.e. cochleostomy). In other studies, cochleostomy was sealed following FM 1-43 FX delivery, often with a permanent cement (i.e. Durelon carboxylate cement); in our study, no such sealing agent could be applied to the RWM following perforation. Thus, following injection, efflux of perilymph from the perforation site occurred as expected for all animals, though this efflux was self-limiting and was no longer present at the 48-hour timepoint. Importantly, despite perilymph efflux, delivery of FM 1-43 FX via microneedle-mediated perforation of the RWM allowed for significant distribution throughout the cochlea, indicating that the majority of the FM 1-43 FX injected through the RWM traversed the inner ear rather than escaping into the middle ear. The cochlear aqueduct is an additional pathway for FM 1-43 FX outflow from the inner ear.

Our polymeric microneedle design was a major strength in that our microneedles were durable enough to sustain both RWM perforation and fluid injection, but maintained their ultrasharp structure to prevent excessive RWM trauma. Additionally, the polymeric material used to construct our microneedles has been shown to be biocompatible and thus may be translated for human trials.

While the disclosed subject matter is described herein in terms of certain non-limiting exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments. In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of non-limiting example embodiments of the disclosed subject matter has been presented for purposes of illustration and description. It is not intended to be exhaustive or to limit the disclosed subject matter to those embodiments disclosed herein

It will be apparent to those skilled in the art that various modifications and variations can be made in the method and system of the disclosed subject matter without departing from the spirit or scope of the disclosed subject matter. While the disclosures herein are described for accessing the cochlea, the issue of localized drug delivery across anatomic barriers to enclosed spaces extends beyond the field of otolaryngology

What is claimed:

1. A method for injecting a therapeutic into the inner ear of a subject, comprising:
providing a microneedle having a sharpened distal tip and a shaft with a maximum outer diameter of about 75 microns to about 150 microns,
the microneedle defining an inner lumen with a diameter about 15 microns to about 50 microns,
the inner lumen having a curvature near the sharpened distal tip such that the inner lumen opens at a side of the microneedle proximal to the sharpened distal tip,
the microneedle mounted on a blunt metallic syringe needle, and
the blunt metallic syringe needle attached to a syringe secured to a micropump;
advancing the microneedle into the middle ear space of the subject and perforating the round window membrane (RWM) of the subject with the sharpened distal tip;
extending the microneedle such that the inner lumen is disposed in the inner ear space of the subject;
injecting a volume of therapeutic into the inner ear space; and
retracting the microneedle from the RWM.

2. The method of claim 1, wherein the microneedle has a maximum outer diameter less than about 100 microns along a portion of the shaft.

3. The method of claim 1, wherein the microneedle is fabricated from a biocompatible polymer, stainless steel, or titanium.

4. The method of claim 1, wherein the blunt metallic syringe needle is a 30 gauge syringe needle.

5. The method of claim 1, wherein the inner lumen has a diameter of 35 μm or less.

6. The method of claim 1, wherein injecting a volume of therapeutic comprises injecting 1.0 μL or less of therapeutic into the inner ear space.

7. The method of claim 6, wherein injecting a volume of therapeutic comprises injecting 1.0 μL or less of therapeutic into the inner ear space with a rate of 1 μL/min or less.

8. A method for injecting a therapeutic into the cochlea of a subject, comprising:
providing a microneedle having a sharpened distal tip and a shaft with a maximum outer diameter of about 75 microns to about 150 microns,
the microneedle defining an inner lumen with a diameter about 15 microns to about 50 microns,
the inner lumen having a curvature near the sharpened distal tip such that the inner lumen opens at a side of the microneedle proximal to the sharpened distal tip, and
the microneedle mounted on a blunt metallsic syringe needle; the blunt metallic syringe needle attached to a syringe secured to a micropump;
advancing the microneedle into the middle ear space of the subject and perforating the round window membrane (RWM) of the subject with the sharpened distal tip;
extending the micro needle through the RWM such that the inner lumen is disposed in the inner ear of the subject;
injecting a volume of therapeutic into the cochlea to distribute the therapeutic into the basal, middle and apical turns of the cochlea; and
retracting of the microneedle from the RWM.

9. The method of claim 8, wherein the microneedle has a maximum outer diameter of less than about 100 microns along a portion of the shaft.

10. The method of claim 8, wherein the microneedle is fabricated from a biocompatible polymer, stainless steel, or titanium.

11. The method of claim 8, wherein the blunt metallic syringe needle is a 30 gauge syringe needle.

12. The method of claim 8, wherein the inner lumen has a diameter of 35 μm or less.

13. The method of claim 8, wherein injecting a volume of therapeutic comprises injecting 1.0 μL or less of therapeutic into the cochlea.

14. The method of claim 13, wherein injecting a volume of therapeutic comprises injecting 1.0 μL or less of therapeutic into the cochlea at a rate of 1 μL/min or less.

15. A system for injecting a therapeutic into the inner ear of a subject, comprising:
a microneedle having a sharpened distal tip and a shaft with a maximum outer diameter of about 75 microns to about 150 microns,
the microneedle defining an inner lumen with a diameter about 15 microns to about 50 microns,
the inner lumen having a curvature near the sharpened distal tip such that the inner lumen opens at a side of the microneedle proximal to the sharpened distal tip,
the microneedle mounted on a blunt metallic syringe needle,
the blunt metallic syringe needle being attached to a syringe secured to a micropump configured to inject 1.0 μL or less of therapeutic into the inner ear at a rate of 1 μL/min or less.

16. The system of claim 15, wherein the microneedle has a maximum outer diameter of less than about 100 microns along a portion of the shaft.

17. The system of claim 15, wherein the microneedle is fabricated from a biocompatible polymer.

18. The system of claim 15, wherein the microneedle is fabricated from stainless steel or titanium.

19. The system of claim 15, wherein the blunt metallic syringe needle is a 30 gauge syringe needle.

20. The system of claim 15, wherein the inner lumen has a diameter of 35 μm or less.
